(19) United States
(12) Patent Application Publication
(10) Pub. No.: US 2006/0008529 A1
(43) Pub. Date: Jan. 12, 2006

(54) Use of additive sites to control nitric oxide release from nitric oxide donors contained within polymers

(76) Inventors: Mark E. Meyerhoff, Ann Arbor, MI (US); Melissa M. Reynolds, Ann Arbor, MI (US); Zhenghong Zhou, Ann Arbor, MI (US)

Correspondence Address: JULIA CHURCH DIERKER;DIERKER & ASSOCIATES, P.C., 3331 W. BIG BEAVER RD. SUITE 109, TROY, MI, 48084-2813, US

(21) Appl. No.: 10/889,646

(22) Filed: Jul. 12, 2004

(51) Int. Cl. A61K 9/14; A61K 31/21

(52) U.S. Cl. 424/486; 514/509

(57) ABSTRACT

A method for increasing, prolonging, and/or controlling the release rates of nitric oxide (NO) from polymeric materials containing NO adducts. Such NO-containing polymeric materials may find use in devices such as blood contacting devices, and biocompatible devices utilizing the same. The method and device utilizes anionic site additives, acidic site additives and/or acidic producing site additives in a polymer that contains NO-adducts to generate higher fluxes of NO to exceed NO threshold levels desirable to substantially prevent and/or minimize reactions such as platelet activation or adhesion.

### STATEMENT REGARDING FEDERALLY SPONSORED RESEARCH OR DEVELOPMENT

This invention was made in the course of research partially supported by a grant from the Department of Health and Human Services (Small Business Innovation Research (SBIR) Grant No. 1 R43 HL072624-01. The U.S. government has certain rights in the invention.

### BACKGROUND

Embodiments of the present invention are directed to nitric oxide donors contained within polymer systems, and methods for forming and using the same.

Nitric oxide (NO) has been shown to have several important physiological functions, including its unique vasodilating properties, cancer-fighting potency, and anti-platelet activity. Although NO is a stable radical, it may be highly reactive with hemoglobin and oxygen, thus making delivery of NO to the target site challenging. Stable hydrophilic, as well as hydrophobic NO donors may be best to take advantage of the potency of NO for a wide range of biomedical applications. These include NO-releasing pharmaceuticals and the preparation of thromboresistive hydrophobic polymeric coatings for medical devices such as intravascular sensors and extracorporeal circuits (based on NO's antiplatelet activity). Indeed, many advances have been achieved using water-soluble diazeniumdiolates as NO delivery agents. For example, the diazeniumdiolate proline (PROLI/NO), when infused into blood, has been shown to relieve muscle spasms. In addition, it has been reported that dimethylene triamine (DETA/NO) diazeniumdiolates substantially suppress overproliferation of cells after vascular injury, and glycosylated diazeniumdiolates possess anti-tumor activity.

However, the use of such water-soluble diazeniumdiolates with hydrophobic matrices to form biocompatible coatings may be problematic. For example, (Z)-1-[N-methyl-N-[6-(N-methylammoniohexyl)amino]]-diazen-1-ium-1,2-diolate (MAHMA/NO) dispersed in a silicone rubber matrix may, in some instances, prevent thrombus formation on the surface of intravascular sensors. The same compound may greatly reduce platelet activity when employed within a polymer coating on the inner walls of extracorporeal circuits. However, MAHMA/NO and its corresponding diamine precursor tend to leach from the surface of the polymer matrix and back react with an oxidative intermediate of NO to form potentially toxic nitrosamines.

In view of this, despite the benefits of NO, the use of NO donors in polymeric systems has been limited.

### SUMMARY

Embodiments of the present invention substantially solve the drawbacks enumerated above by providing a biocompatible material. The biocompatible material has a polymer matrix with a nitric oxide adduct therein that is capable of releasing NO. The nitric oxide adduct may be dispersed within the polymer matrix and/or covalently attached thereto, depending on the type of adduct used. Within the polymer matrix exists at least one of an anionic site additive, an acidic site additive, and/or a site adapted to produce an acidic site additive within the polymer matrix. The additive(s) are adapted to increase, prolong, and/or control NO release rates from the NO-donors.

### BRIEF DESCRIPTION OF THE DRAWINGS

Objects, features and advantages of embodiments of the present invention will become apparent by reference to the following detailed description and drawings, in which:

FIG. 1 is a graph of UV absorbance spectra as a function of time for compounds 1a-1e in PBS buffer at pH 7.4;

FIG. 2 is a graph depicting total NO release curves for compounds 2a and 2d (equal molar ˜5 wt. %) dispersed in a 1:2 PVC/DOS polymer matrix (circular disks with a diameter of 8 mm and a thickness of ˜150 μm) as a function of time in PBS buffer at 37° C., NO was measured directly by chemiluminescence;

FIG. 3A is a graph depicting NO surface flux for compound 2d dispersed in a 1:2 PVC/DOS matrix (circular disks with a diameter of 8 mm and a thickness of ˜150 μm) with and without KTpClPB (1:1 mol ratio KTpClPB:compound 2d) soaked in PBS buffer (pH 7.4) at 37° C., NO was measured directly by chemiluminescence;

FIG. 3B is a graph depicting total NO release curves for compound 2d dispersed in a 1:2 PVC/DOS matrix (circular disks with a diameter of 8 mm and a thickness of ˜150 μm) with and without KTpClPB (1:1 mol ratio KTpCIPB:compound 2d) soaked in PBS buffer (pH 7.4) at 37° C., NO was measured directly by chemiluminescence;

FIG. 4 is a schematic view depicting that the presence of potassium tetrakis(4-chlorophenylborate (depicted as K⁺B⁻) reduces pH changes within polymeric films containing N-diazeniumdiolates;

FIG. 5A is a graph depicting the visible spectra of 9-dimethylamino-5-[4-(16-butyl-2,14-dioxo-3,15-dioxaeicosyl)pneynylimino]benzo[a]penoxazine (chromoionohpore II) incorporated into a 1:2 PVC/DOS matrix containing compound 2d soaked in PBS buffer pH 7.4 as a function of time with KTpClPB, the protonated absorption peak of chromoionophore II is at 650 nm and the deprotonated band is at 514 nm;

FIG. 5B is a graph depicting the visible spectra of chromoionohpore II incorporated into a 1:2 PVC/DOS matrix containing compound 2d soaked in PBS buffer pH 7.4 as a function of time without KTpClPB, the protonated absorption peak of chromoionophore II is at 650 nm and the deprotonated band is at 514 nm;

FIG. 6A is a graph depicting the NO surface flux for compound 2d dispersed in plasticized PVC films (ratios 1:2, 1:1 and 2:1 PVC:DOS by mass) containing KTpClPB in PBS buffer (pH 7.4) at 37° C., NO was measured directly by chemiluminescence;

FIG. 6B is a graph depicting total NO release curves for compound 2d dispersed in plasticized PVC films (ratios 1:2, 1:1 and 2:1 PVC:DOS by mass) containing KTpClPB in PBS buffer (pH 7.4) at 37° C., circular films of 8 mm in diameter and ˜150 μm thickness were used, NO was measured directly by chemiluminescence;

FIG. 7A is a graph depicting NO surface flux for compound 2d dispersed in a SR/DOS matrix containing KTpClPB in PBS buffer (pH 7.4) at 37° C., SR tubing (diameter ˜2 mm) was dip-coated in a solution including 81.2 wt. % SR, 6.5 wt. % DOS, 4.2 wt. % compound 2d and 8.1 wt. % KTpClPB, NO was measured directly by chemiluminescence;

FIG. 7B is a graph depicting total NO release curves for compound 2d dispersed in a SR/DOS matrix containing KTpClPB in PBS buffer (pH 7.4) at 37° C., SR tubing (diameter ˜2 mm) was dip-coated in a solution including 81.2 wt. % SR, 6.5 wt. % DOS, 4.2 wt. % compound 2d and 8.1 wt. % KTpClPB, NO was measured directly by chemiluminescence;

FIG. 8A is a graph depicting NO surface flux for compound 2d dispersed in 1:2 PVC/NPOE and PVC/DOS matrices containing KTpClPB in PBS buffer (pH 7.4) at 37° C., circular disks having 8 mm diameter and ˜150 μm thickness were used, NO was measured directly by chemiluminescence;

FIG. 8B is a graph depicting total NO release curves for compound 2d dispersed in 1:2 PVC/NPOE and PVC/DOS matrices containing KTpClPB in PBS buffer (pH 7.4) at 37° C., circular disks having 8 mm diameter and ˜150 μm thickness were used, NO was measured directly by chemiluminescence;

FIG. 9A is a graph depicting NO surface flux for 4 wt. % and 8 wt. % compound 2d dispersed in a 1:2 PVC/DOS matrix containing KTpClPB (1:1 mol ratio with compound 2d) in PBS buffer (pH 7.4) at 37° C., circular disks having 8 mm diameter and ˜150 μm thickness were used, NO was measured directly by chemiluminescence;

FIG. 9B is a graph depicting total NO release curves for 4 wt. % and 8 wt. % compound 2d dispersed in a 1:2 PVC/DOS matrix containing KTpCIPB (1:1 mol ratio with compound 2d) in PBS buffer (pH 7.4) at 37° C., circular disks having 8 mm diameter and ˜150 μm thickness were used, NO was measured directly by chemiluminescence;

FIG. 10A are images of a control polymer (plasticized PVC) coated VECTRA™ vascular access grafts after removal from 21 d implantation in sheep;

FIG. 10B are images of a polymer (plasticized PVC) containing compound 2d coated VECTRA™ vascular access grafts after removal from 21 d implantation in sheep;

FIG. 11A are representative histology images of control VECTRA™ vascular access grafts after removal from 21 d implantation in sheep at magnifications of 10× and 20×;

FIG. 11B are representative histology images of compound 2d coated VECTRA™ vascular access grafts after removal from 21 d implantation in sheep at magnifications of 10× and 20×;

FIG. 12A is a graph depicting NO surface flux for polymethacrylate-based diazeniumdiolate embedded in 2:1 PVC/DOS matrix (circular disks with a diameter of 7 mm and a thickness of ˜150 mm, top coated with PVC) with and without PLGA (10% wt, lactide:glycolide (50:50), average Mw 50,000-75,000 ) soaked in PBS buffer (pH 7.4) at 37° C., NO was measured directly by chemiluminescence; and

FIG. 12B is a graph depicting total NO release for polymethacrylate-based diazeniumdiolate embedded in 2:1 PVC/DOS matrix (circular disks with a diameter of 7 mm and a thickness of ˜150 mm, top coated with PVC) with and without PLGA (10% wt, lactide:glycolide (50:50), average Mw 50,000-75,000 ) soaked in PBS buffer (pH 7.4) at 37° C., NO was measured directly by chemiluminescence.

### DETAILED DESCRIPTION

NO may prevent both platelet activation and aggregation, and thus polymeric materials doped with lipophilic NO donors (for example, diazeniumdiolate type donors) are attractive with respect to preparing more blood-compatible polymer materials. However, in order to effectively use NO donors in these systems, the NO donor should remain stable through the preparation process of embedding the donor within the polymer matrix, and should further be capable of spontaneously releasing NO when the polymer is exposed to solutions and/or blood under physiological conditions. However, previously known discrete diazeniumdiolates have NO releases dependent upon pH and temperature, which may, in some instances, cause the NO donor to be unstable, incapable of spontaneous release, and/or unable to release NO at a prescribed rate for a long enough period of time.

Disclosed herein are biocompatible materials having anionic and/or acidic sites and NO-adducts and devices utilizing the same. Suitable biocompatible materials include various polymers having anionic and/or acidic site additives and containing NO-adducts/donors capable of generating fluxes of NO in sufficient concentrations to prevent and/or minimize adverse physiological interaction. Also disclosed is a method for making such biocompatible materials.

It has been found that the NO release characteristics of these compounds alone, as well as within a polymer matrix under physiological conditions, are advantageously controllable. Further, the resulting materials may advantageously be used as thromboresistant coatings for vascular grafts.

As disclosed herein, biocompatible materials having anionic site additives and/or acidic site additives and/or a site(s) adapted to produce an acidic site additive within the polymer matrix may be employed to provide NO release in a manner that prevents undesirable interactions including, but not limited to, platelet activation and/or adhesion, improper vasodilation, and/or undesirable cell proliferation, such as proliferation of smooth muscle cells.

“Nitric oxide adducts” (NO adducts) and “NO-donors” refer to compounds and functional groups which, under physiological conditions, can donate and/or release NO such that biological activity of the NO is expressed at the intended site of action/target site.

An embodiment of the biocompatible material is a polymeric system containing NO adducts/donors. The NO adduct may be integrated into the polymeric system in any suitable manner, a non-limitative example of which is doping. Suitable NO adducts (non-limitative examples of which include lipophilic adducts and discrete adducts) are generally those exhibiting capability of embedding (either by covalent attachment and/or dispersion) into the polymer matrix and exhibiting process preparation stability. “Lipophilic NO adducts” as referred to herein are those NO adducts (a non-limitative example of which are diazeniumdiolates) which, when placed into a polymer matrix, release therapeutic amounts, ranging between about 1% and about 100%, of NO from the polymer phase. “Discrete NO adducts” as referred to herein are those compounds that have the NO-releasing moiety covalently attached to a small molecule or to a polymer filler (e.g., functionalized silica particles or titanium particles). It is to be understood that discrete NO adducts are generally not polymers. Those compounds that have their NO-releasing moiety covalently attached to a polymer backbone are generally referred to as “polymeric NO adducts.” Non-limitative examples of suitable polymeric NO adducts include, but are not limited to, diazeniumdiolated silicone rubbers (DACA/N₂O₂), diazeniumdiolated methacrylates, diazeniumdiolated polyurethanes, diazeniumdiolated poly(vinyl chloride), and/or mixtures thereof. It is to be understood that generally neither the discrete NO adducts nor the polymeric NO adducts has a protecting group(s) attached thereto. However, in an embodiment in which the discrete NO adducts and/or polymeric NO adducts have a benign protecting group, it is to be understood that when the protecting group is released, a benign species is yielded. Still further, the benign protecting group of an NO adduct or a polymeric adduct may be removed prior to and/or during NO release. Furthermore, if a protecting group is utilized that is non-benign, it is to be understood that the protecting group is removed prior to application of the device (e.g. NO release).

Examples of suitable benign protecting groups include, but are not limited to sugar or sacharride protecting groups (e.g. glycosylated protecting groups that contain glucose, galactose, or mannose), glycosylated protecting groups that are derivatized sugar protecting groups (e.g. aceylated glucose, galactose, or mannose), and/or mixtures thereof. Specific non-limitative examples of the sugar protecting groups include O₂—B-Galactosepyranosyl and O₂-a-D-Mannopyranosyl.

Examples of suitable non-benign protecting groups include, but are not limited to O₂-vinyl groups, O₂-acetoxymethyl groups, and/or mixtures thereof. Specific non-limitative examples of non-benign protecting groups include O₂-aryl derivatives such as O₂-[2,4-dinitrophenyl], O₂-[2-Nitro-4(trifluoromethyl)phenyl], O₂-[3-nitropyrid-2yl], 1-(2-Bromoethoxy), and/or mixtures thereof.

The NO adduct of choice is also one capable of spontaneous release of NO when the polymer is exposed to solutions and/or blood under physiological conditions. Some non-limitative examples of NO adducts include protected and discrete N-diazeniumdiolates, nitrosothiols, organic nitrates, metal-nitrosyls, C-based diazeniumdiolates, and/or mixtures thereof.

Spontaneous release of NO from the polymer may be governed by at least one process occurring between the NO adduct and the aqueous environment. These include, but are not limited to at least one of diffusion and ionization of water into/within the organic polymer; ion-exchange between the buffer ions and ions within the polymer; protonation of amine-nitrogen-bearing compounds to yield NO; and deprotonation of water by secondary amine sites to yield organic ammonium hydroxides. Suitable nitrogen-bearing compounds include, but are not limited to, various diazeniumdiolates.

Various hydrophobic polymer materials may be employed in the material, method, and device as disclosed herein. These include, but are not limited to materials such as poly(vinyl chloride) (PVC), silicone rubbers (SR), polyurethanes (PU), polymethacrylates, polyacrylates, polycaprolactones, polylactide, polyglycolide, poly(lactide-co-glycolide), copolymers thereof, and/or mixtures thereof. The polymer of choice will be one capable of releasing NO from, for example, covalently attached and/or dispersed diazeniumdiolate type NO-adducts.

It is to be understood that discrete nitric oxide adducts may be either covalently attached to the polymer matrix or may be dispersed therein. Some examples of discrete diazeniumdiolates include, but are not limited to anionic diazeniumdiolates stabilized with metal cations, zwitterionic diazeniumdiolates, and protected discrete diazeniumdiolates (e.g. O² protected discrete diazeniumdiolates). In an embodiment incorporating protected nitric oxide adducts (such as protected N-diazeniumdiolates), it is to be understood that the protected nitric oxide adducts may be dispersed substantially throughout the polymer matrix.

The polymer may be doped with suitable anionic sites and/or acidic sites. Anionic site additives, as referred to herein, is defined as compounds or inherent polymer compositions that act as a buffer in the polymer/organic phase to minimize or substantially prevent pH changes in the polymer film containing the NO donor. Examples of suitable anionic site additives include, but are not limited to salts, non-limitative examples of which include potassium tetrakis-4-(chloro)phenyl borate, sodium cholate, carboxylated poly(vinyl chloride), dinonylnaphthalene sulfonate (DNNS), phosphatidylglycerol, L-phosphatidic acid, L-glycerol 3-phosphoric acid, phosphoglycerides, phosphatidylinsitol, sodium salts, potassium salts or other salts, cholesterols, steroid derivatives, lipids, phosphatidyl chlorine, prostaglandins, lipophilic fatty acids, lipophilic sugars, and/or mixtures thereof. It is to be understood that many of these anionic site additives are naturally occurring in the blood and/or the contacted surface, and thus are advantageously not toxic to the blood and/or contacted surface if they leach out of the polymer matrix. An embodiment of the biocompatible material may also be prepared with polymers containing inherent (naturally occurring) anionic sites (e.g., carboxylated poly(vinyl chloride) and a sodium salt of carboxylated poly(vinyl chloride)). Some further non-limitative examples of polymers having inherent anionic sites include polymers with —COOH(Na), —SO₃H (or Na), —NHSO₃H (or Na) functional groups, and/or mixtures thereof, for example, PVC—COOH, polymethacrylic acid, poly(anetholesulfonic acid, sodium salt).

It is contemplated that without the use of anionic sites for N,N′-dibutylhexamethylenediamine diazeniumdiolate dispersed within a plasticized PVC matrix, the level of NO release rapidly decreases. Without being bound to any theory, it is believed that the use of the anionic sites added to, or inherent within, the polymeric material minimizes pH changes in the polymer matrices (pH changes affect the kinetics of decomposition of most NO-donors). In some cases, the incorporation of anionic sites may be accomplished by adding a salt to a polymer in organic solution. In other cases, the salt may be added in the processing stage, for example, when the tubing or thin films of such polymer coatings are molded or cast, respectively, from the native polymer material. In an embodiment, polymers containing inherent anionic sites may be dissolved in organic solution and the NO-donor incorporated into the matrix. This allows the ions to diffuse from the polymer matrix to the surrounding aqueous phase, and NO release may advantageously be maintained at a relatively constant rate until the total concentration of the diazeniumdiolate NO-donor species decreases significantly.

Non-limitative examples of NO donors used to prepare an embodiment of the biocompatible material having anionic and/or acidic site additives capable of providing controlled NO release rates from NO-donors are diazeniumdiolates derived from dialkyl hexamethylene diamine compounds (parent structures 1a-e where R corresponds to those listed in Table 1) having the general linear structure: 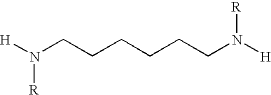
to form corresponding N-diazeniumdiolate (2a-e) derivatives having the general formula: 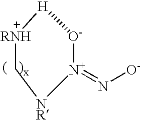
in which R is an alkyl group having one to twelve carbon atoms or a branched side chain. It is to be understood that the R groups may be different in character. For example, one R group may be a propyl group while another R group may be a butyl group. In an embodiment, the R groups may be hydrogen. Still further, the methylene spacer present between the amines in the derivatives may range from x=1 to x=6.

Other non-limitative examples of parent structures used to form diazeniumdiolates may be any primary or secondary amine containing compounds, including, but not limited to: 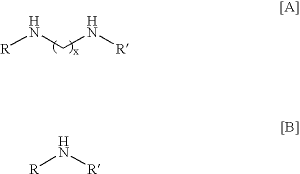
where R and R′ may be hydrogen; n-alkyls; branched alkyls; aliphatics; cyclic and/or aromatic amine side-chains; ketones; aldehydes; amides; ether; esters; alkenes; alkynes; and/or mixtures thereof; and/or the like. Examples of the diazeniumdiolates that may be formed from parent structure A include the following:

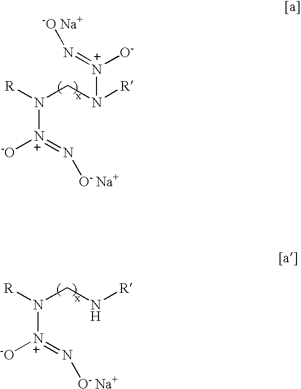

Examples of the diazeniumdiolates that may be formed from parent structure B include the following:

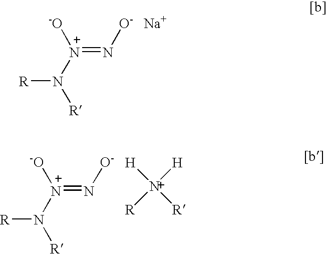

As a non-limitative example, a sodium ion is depicted in structures a, a′, b, and b′ as a counter ion in order to stabilize the respective diazeniumdiolates. It is to be understood that other metal ions such as ions of lithium, potassium, copper, and/or the like, and/or mixtures thereof, may be valid metal cations to stabilize the species.

As depicted, anionic diazeniumdiolates with the previously mentioned diamine backbone or compounds containing one amine site or those containing three or more amine sites may be used in an embodiment of the present invention.

In one embodiment, the NO release from polymer matrices containing dispersed diazeniumdiolates, covalently attached discrete diazeniumdiolates, or polymeric diazeniumdiolates may be enhanced by incorporating one or more sites adapted to produce an acidic site additive within the polymer matrix (non-limitative examples of which include biodegradable polymers/copolymers (e.g. PLGA: poly(lactide-co-glycolide) and compounds whose decomposition products produce hydronium ions or water) to a hydrophobic polymer matrix). Suitable polymers generating acidic sites may generally be recited as polymers with ester linkages, and/or other linkages which undergo hydrolysis under physiological conditions to generate acidic sites. Some non-limitative examples of such polymers adapted to generate acidic sites include polylactide, polyglycolide, polycaprolactone, poly(lactide-co-glycolide), poly(lactide-co-caprolactone), and/or mixtures thereof.

It is to be understood that an acidic site additive may be directly added to the polymer matrix, or may be a site capable of producing an acidic site additive within the polymer matrix may be added. It is to be further understood that the acidic site additives/acidic site producing additives may be added in place of, or in addition to, the anionic site additives. In an embodiment, polymer/copolymers having uncapped acidic end groups or polymers/copolymers having acidic groups on the backbones/pendant side chains may be used as the acidic site additives. In an alternate embodiment, it has been advantageously found that the ester linkage of a polymer/copolymer acidic site producing additive may be hydrolyzed in the aqueous environment of the body to generate acidic microclimate within the polymer matrix, as shown in the following scheme:

The hydrolysis of poly(lactide-co-glycolide) in the aqueous environment.

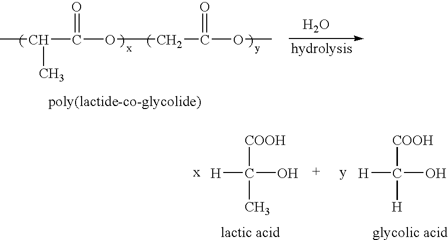

The presence of this reaction may advantageously diminish the increase of pH (high pH inhibits NO generation) caused by the decomposition of diazeniumdiolate to release NO. Without being bound to any theory, it is believed that by using PLGA with either variant lactide/glycolide ratios or different molecular weight (two important factors controlling the degradation rates), NO flux from the polymer surface may be better controlled. Further, the copolymer additive is generally harmless to the body since the final hydrolytic products are monomers: glycolic acid and lactic acid. Both monomers may enter the tricarboxylic acid cycle and may be eliminated from the body as carbon dioxide and water. As an example of this embodiment, NO release from a plasticized PVC film embedded within a polymethacrylate-based NO donor (shown below) 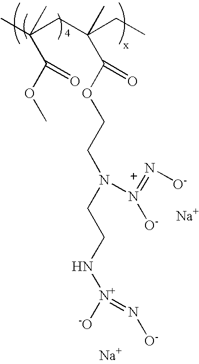
and the biodegradable additive have been enhanced compared to that from the same film without such additive (See FIGS. 12A and 12B).

A non-limitative example of an embodiment of the biocompatible material includes a base polymer layer, one or more intermediate polymer layers, and a top polymer layer. In an embodiment, the top and/or base polymer layers may be made of any suitable polymeric material and/or polymer/plasticizer mixture. It is to be understood that the top and/or base polymer layers may be composed of the same or different polymer/plasticizer compositions. It is to be further understood that the intermediate polymer layer(s) may have the same, similar or a different composition than the base layer, the top layer, and any other intermediate layers employed. Further, the intermediate polymer layer(s) may also contain NO-adducts, anionic site additives and/or acidic site additives in the same, similar or different amounts than the other intermediate layers employed. For example, one intermediate layer may contain an NO adduct, a second intermediate layer may contain an NO adduct and acidic site additives, while a third intermediate layer may contain anionic and acidic site additives. It is to be understood that one or more of the intermediate layers may also contain polymers that do not contain NO donors or additives. These intermediate layers may also be composed of the same polymer material(s) as the other layers (e.g. top and base layers) or they may have a different composition.

Experimental

Synthesis. Previously tested diamines to synthesize diazeniumdiolates typically result in highly water-soluble products. The current inventors have tested the effect of the side alkyl chain length on the addition of NO to lipophilic diamine structures. The parent N-N′-dialkylhexamethlyenediamine structure (1a-g having varying R groups identified in Table 1) and the corresponding N-diazeniumdiolates (2a-g in Table 2) formed upon addition of NO are illustrated above. The length of the R chain, R═CH₃ to R═(CH₂)₁₁CH₃, is systematically varied.

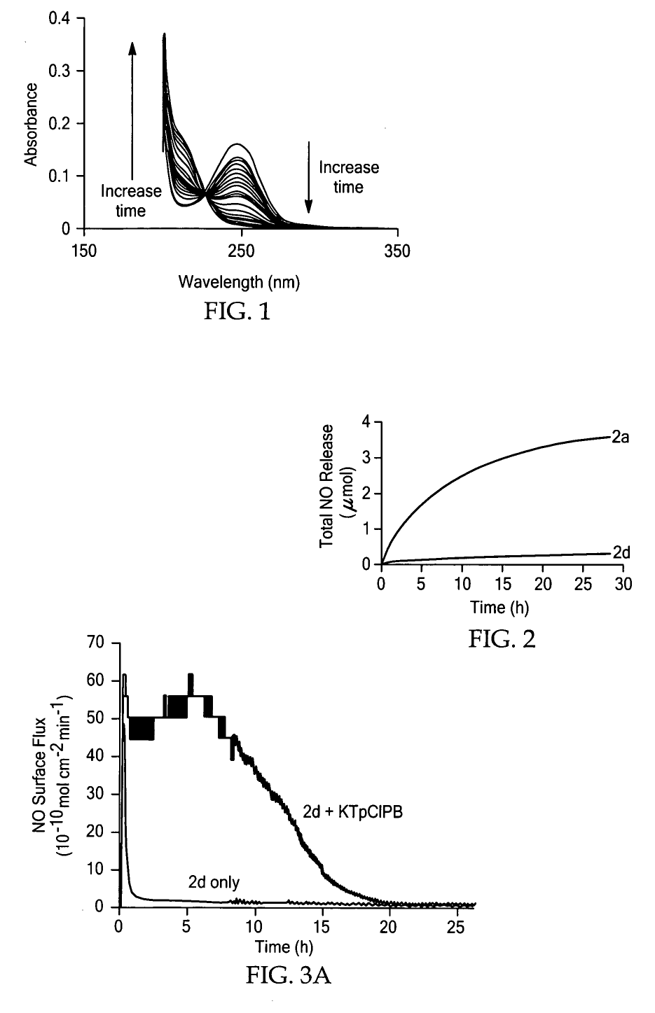

Decomposition of diazeniumdiolates. Diazeniumdiolates have been shown to decompose and release NO by two mechanisms, proton-driven and thermal dissociation. To date, proton-driven decomposition is most prevalent for discrete amine based diazeniumdiolates. UV spectroscopy and NO selective chemiluminescence measurements were used to monitor the decomposition of the diazeniumdiolates with time at pH 7.4. FIG. 1 shows the UV spectra of 1a-e as a function of time in PBS (phosphate buffered saline) buffer. The absorbance maximum is 247 nm for methanol (1d and 1e) or basic solutions (1a-1c) and decreases with time when 1a-1e are exposed to PBS buffer, while there is a corresponding increase in the nitrite absorbance band at 214 mn.

The intramolecular diazeniumdiolates released 2 moles of NO for each mole of diamine (see Table 1). These values were determined using chemiluminescence, after adding a given amount of the diazeniumdiolate to PBS buffer purged with nitrogen. The NO released was detected and integrated over time, until no further release of NO was observed.

As shown in Table 2, it was generally not possible to form air-stable intramolecular diazeniumdiolates from the most lipophilic species, N,N-dihexylhexamethylene diamine (1f) and N,N-didodecylhexamethylene diamine (1g). The reaction of NO with If yielded a diazeniumdiolate (as determined by UV) that was initially air-stable, but decomposed after 12 hours even with storage at −20° C. While the reaction of NO with I g yields a diazeniumdiolated species that can be observed if maintained under a nitrogen environment, the presence of oxygen during the work-up procedure immediately decomposes the most lipophilic intramolecular diazeniumdiolates to corresponding ammonium nitrite salts. Thus, the most air-stable intramolecular diazeniumdiolate that could be isolated was that of diamine 1e. Air stable bis-diazeniumdiolates of 1f and 1g may, however, be prepared when an exogenous base such as sodium methoxide is present in the reaction mixture.

Thermal stability of diazeniumdiolates. To investigate the temperature stability of the various intramolecular diazeniumdiolates, thermal gravimetric analysis was performed on the analogue series. Thermal stability may be important for storage and processing conditions, particularly if such compounds are to be used to prepare polymeric coatings for medical devices. Under a nitrogen atmosphere, the diazeniumdiolates studied remain stable up to about 104° C. (see Table 2) before losing their diazeniumdiolate moiety and leaving only the parent diamine, as confirmed by proton NMR. There appears to be no difference in the thermal stability of the diazeniumdiolates as a function of side chain length under a nitrogen atmosphere. The decomposition at this temperature may be due in part to disruption of the hydrogen bonding interaction between the oxygen of the diazeniumdiolate and the ammonium hydrogen. Based on the percent weight change, the loss of the diazeniumdiolate moiety is observed at a single temperature.

Kinetics. The decomposition of intramolecular zwitterionic diazeniumdiolates have been shown to follow pseudo-first order kinetics. As the pH of the environment becomes more basic, the rate of decomposition to liberate NO decreases. The decomposition of the diazeniumdiolates prepared in this work, at pH 7.4, is also summarized in Table 2. The concentration of diazeniumdiolates was monitored with time in PBS buffer using chemiluminescence, the diazeniumdiolates exhibited decomposition following first order kinetics, in which the plot of the natural logarithm of concentration vs. time yields a linear relationship with r²≧0.99. There is an increase in the “apparent” half-lives as R is increased from —CH₃ to —CH₂CH₃. The term “apparent” half-life is used to refer to the half-lives of those compounds that have limited solubility in PBS buffer (heterogeneous suspensions). As R is further increased from —CH₂CH₃ to —(CH₂)₂CH₃ to —CH₂)₄CH₃, the half-lives decrease slightly from that measured for R═—CH₂CH₃. However, the differences observed in the “apparent” half-lives of 2c-e are within the standard deviation of the measurement, indicating that no substantially clear trend with lipophilicity may be gleaned.

Use in Hydrophobic Systems. To date, the use of NO donors in polymeric systems has been limited despite the well-documented benefits of NO. Because NO can both prevent platelet activation and aggregation, polymeric materials doped with lipophilic NO donors are attractive with respect to preparing more blood compatible polymer materials. However, in order to effectively use NO donors in these systems, the NO donor must remain stable through the preparation process of embedding the donor within the polymer matrix, and must further be capable of spontaneously releasing NO when the polymer is exposed to solutions or blood under physiological conditions. The release of NO from molecules embedded within a polymer matrix has additional variables that may govern NO release profiles from within these materials. Under physiological conditions, the processes that are occurring between the polymer, the embedded NO donor and the aqueous environment include, but are not limited to the diffusion and ionization of water into/within the organic polymer film; ion-exchange between the buffer ions and ions within the polymer; protonation of the amine nitrogen-bearing the diazeniumdiolate to yield NO; and deprotonation of water by secondary amine sites to yield organic ammonium hydroxides.

The effect of organic phase pH on NO-release from polymeric materials. As previously demonstrated, the rate constant for diazeniumdiolate decomposition is pH dependent. Incorporation of 5 wt. % of compound 2d into a PVC film plasticized with dioctyl sebacate (DOS) (approximate thickness between about 150 and about 200 μm), and then exposure to PBS buffer, yielded an NO release profile measured by chemiluminescence. The profile had an initial bolus of NO that decreased rapidly with time, never achieving theoretical total NO release (i.e., the amount of NO anticipated based on the mass of compound 2d in the polymer film) (FIG. 2). This is in sharp contrast to compound 2a which releases all of its NO in a short period of time (FIG. 2). However, the decomposition of compound 2a occurs, partly, outside of the polymer matrix; that is, compound 2a diffuses from the polymer matrix and then reacts with the protons within the soaking solution to release NO. For the more lipophilic NO donors, where the NO is released primarily from water induced protonation within the polymer matrix, additional additives are required to prolong the release from the polymeric films and achieve theoretical NO release (based on the total amount of diazeniumdiolate doped within the material).

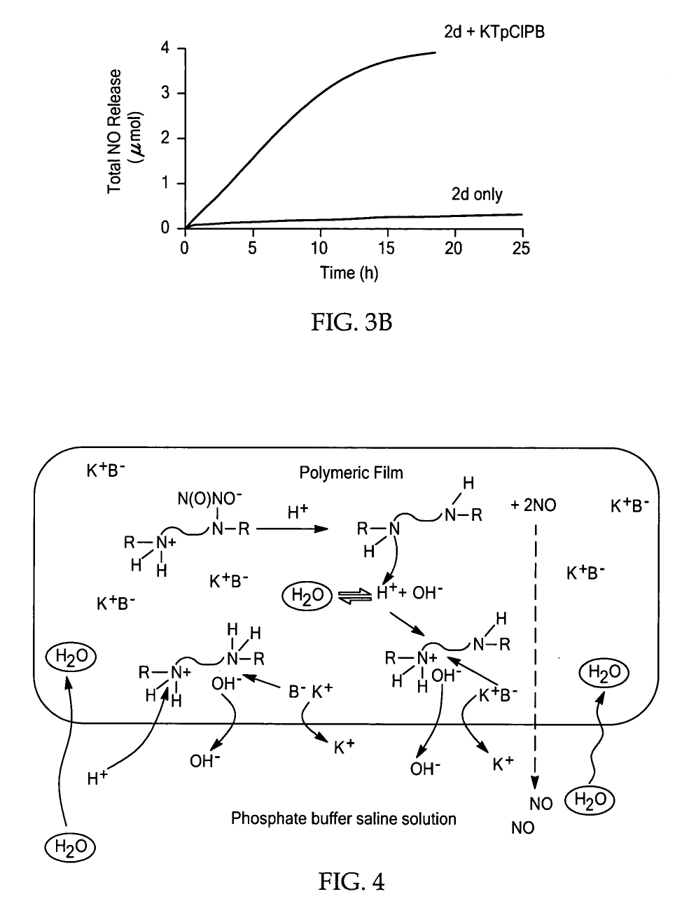

The addition of a lipophilic tetraphenylborate salt (potassium tetrakis-4-chlorophenyl borate (KTpClPB)) into the polymer matrix increases, prolongs, and helps to control the release of NO from plasticized PVC films containing 2d (FIGS. 3A and 3B). In the absence of the KTpClPB, NO release decreases dramatically after approximately 1 hour. Similar NO release patterns are also observed for 2c and 2e dispersed within a plasticized PVC matrix. The decrease in NO release observed is believed to result from an increase in the pH within the organic polymer film, which decreases the decomposition rate of lipophilic diazeniumdiolates. As water diffuses into the film initiating NO release, secondary amine sites result. The secondary amine sites have a higher pKa than water and therefore deprotonate water to form hydroxide ions. The basic microdomain environment that results within the polymer, in turn, slows further decomposition of the remaining diazeniumdiolates that would generate NO. This retardation may occur as a result of the organic ammonium hydroxide microphases within the polymer, which in turn serves to stabilize the remaining diazeniumdiolates.

The incorporation of KTpClPB buffers the polymer phase by providing lipophilic anionic sites that may serve as counterions to the organic ammonium cations as depicted schematically in FIG. 4. The potassium and hydroxide ions may diffuse from the polymer matrix to the surrounding aqueous phase and NO release is maintained at a more constant rate until the total concentration of diazeniumdiolate species decreases significantly.

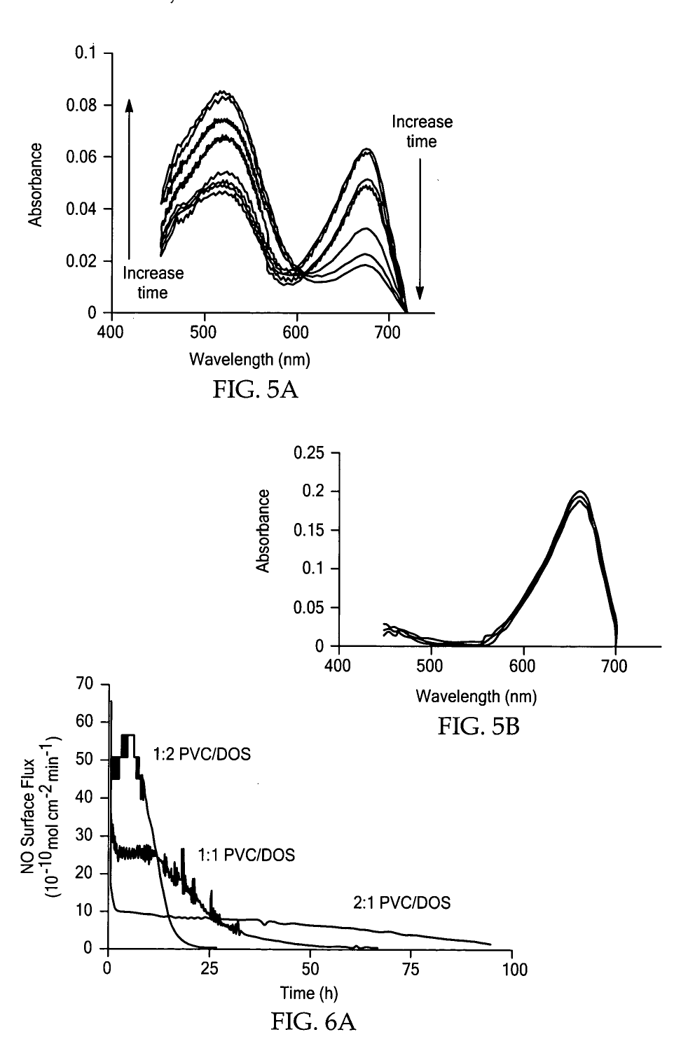

To determine, experimentally, if the pH within the film was changing with time, a lipophilic pH chromoionophore was incorporated into PVC films containing 5 wt. % of 2d, both in the presence and absence of KTpClPB. The intensity of the UV-Vis absorbances corresponding to the protonated (λₘₐₓ=650 nm) and deprontonated (λₘₐₓ=514 nm) peaks of the chromoionophore were monitored with time spectrophotometrically. It was found that films without KTpClPB showed an increase in the absorbance of the deprotonated peak and a decrease in the protonated peak as a function of exposure time to the PBS buffer (FIG. 5A). In contrast, once equilibrium is established, no change is observed in the ratio of the protonated and deprotonted peaks and primarily only a protonated species is observed for films containing KTpClPB where the intensity of the protonated peak remains constant (FIG. 5B). This further supports the notion that as NO is being released from within the polymer phase, the unreacted diazeniumdiolate groups may require a buffering system to prevent the film from becoming too basic, which in turn may slow further diazeniumdiolate decomposition.

The effect of polymer matrix on NO-release. Diazeniumdiolates of similar structure to those investigated herein decompose to generate NO by a primarily proton driven mechanism. Thus, polymer matrices that favor water or proton partitioning and diffusion within the matrix should provide the fastest NO release profiles. By formulating different polymer compositions, thereby yielding different water up-take amounts and diffusion coefficients of reacting species, the NO release profiles of a diazeniumdiolate in a particular polymer matrix may be controlled.

It has been previously reported that the water up-take values for plasticized PVC films are dependent on the matrix composition, and as the polymer to plasticizer ratio is increased, the water uptake values decrease. This decrease in the water up-take may lead to a lower proton activity within the polymer matrix as the polymer to plasticizer ratio is increased. In addition, the diffusion coefficients of species in a plasticized polymer decreases with lower plasticizer content. The coupling of lower proton activity with decreased diffusion coefficients as the polymer to plasticizer ratio increases may lead to a dramatic decrease in the rate constant for the decomposition of the diazeniumdiolate due to a decrease in the probability of the reacting species (the diazeniumdiolated N and the proton) to collide and liberate NO. Thus, a lower flux of NO from films with higher polymer to plasticizer ratios would be predicted.

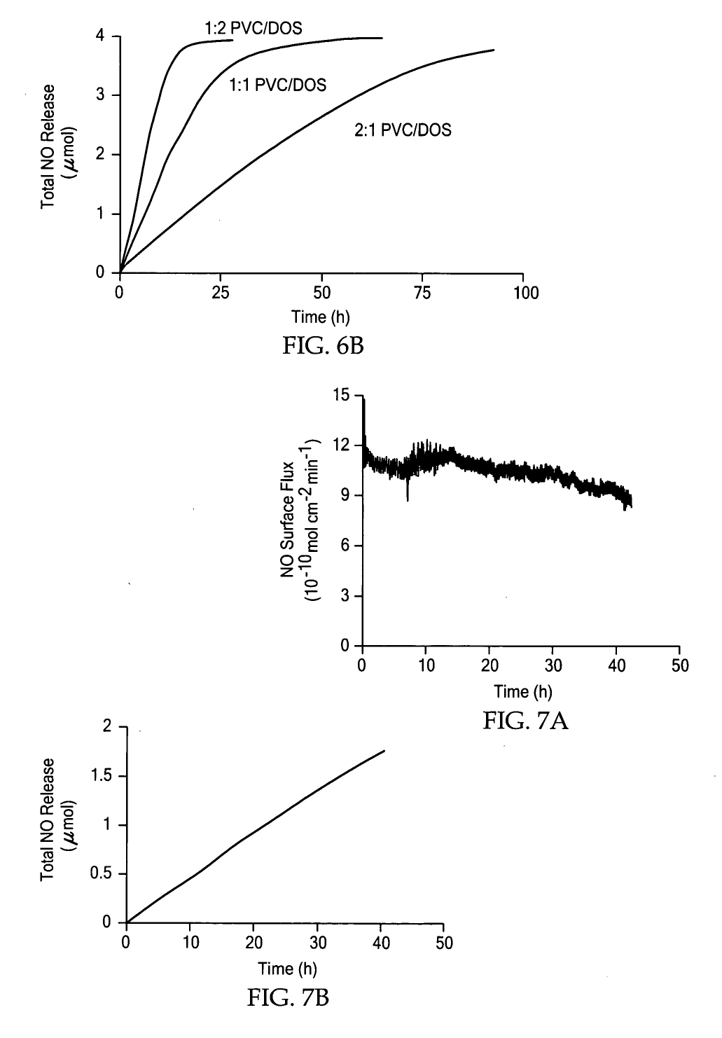

Indeed, as shown in FIG. 6B, as the polymer to plasticizer ratio is increased from 1:2 to 2:1 for films containing compound 2d and KTpClPB, the NO release rate decreases and the release is prolonged. For each variation in the polymer to plasticizer ratio, there exists a linear region of NO release. As the polymer to plasticizer ratio is increased, the time over which the NO release is steadily lengthened. This is especially advantageous for applications that require constant NO release for longer periods of time. Blends with even higher polymer to plasticizer ratios may have even more prolonged release that may be very useful for certain medical applications that require continued NO generation over several days (i.e., implantable sensors, extracorporeal membrane oxygenation (ECMO), vascular grafts, etc.).

One polymer blend in which this prolonged high steady surface flux of NO has been observed is silicone rubber (SR) (FIG. 7A). As shown in FIG. 7B, the NO released from silicone rubber tubes coated with a solution of plasticized SR with 4.2 wt. % compound 2d and 8.1 wt. % KTpClPB is steady over a 40 h period, with only 15% of the total estimated NO released from the surface of the tube after this time upon exposure to PBS buffer at 37° C. This release may continue for weeks. The prolonged NO release is believed, without being bound to any theory, to be a result of the relatively low water uptake of the SR matrix coupled with the buffering effect of having the lipophilic borate salt present as well.

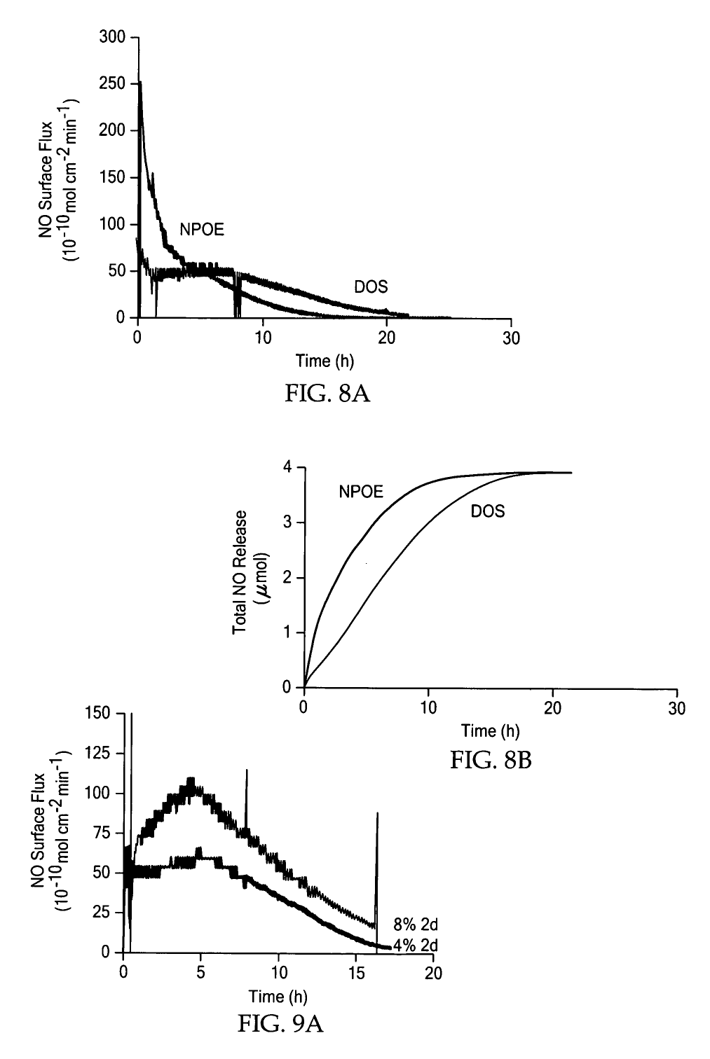

Another approach that may be used to alter the NO release profile of diazeniumdiolates from a polymer matrix is to change the plasticizer. Plasticizers are often blended with polymers to make the matrix more flexible and promote diffusion of species within the material (e.g., ion selective electrodes). To examine the effect of plasticizer on NO release from PVC films, two plasticizers with different dielectric constants were used: o-nitrophenyloctyl ether (NPOE) and dioctyl sebacate (DOS). The formulation of the polymer matrix (i.e., ratio of polymer to plasticizer, incorporation of KTpClPB) remained constant. By using a more polar plasticizer such as NPOE (dielectric constant (ε) of 21), water partitions into the polymer more favorably, leading to an increased source of protons, and therefore a faster rate of NO release. However, the intrinsic pKa of the amine that possesses the diazeniumdiolate group may increase (become more basic) in matrices prepared with the more polar plasticizer. This may lead to faster NO release from polymer matrices containing a more polar plasticizer. Indeed, changing the plasticizer type does alter the NO release profile, as shown in FIG. 8B, where higher initial NO release is observed for compound 2d incorporated into a PVC/NPOE matrix compared with a PVC/DOS matrix.

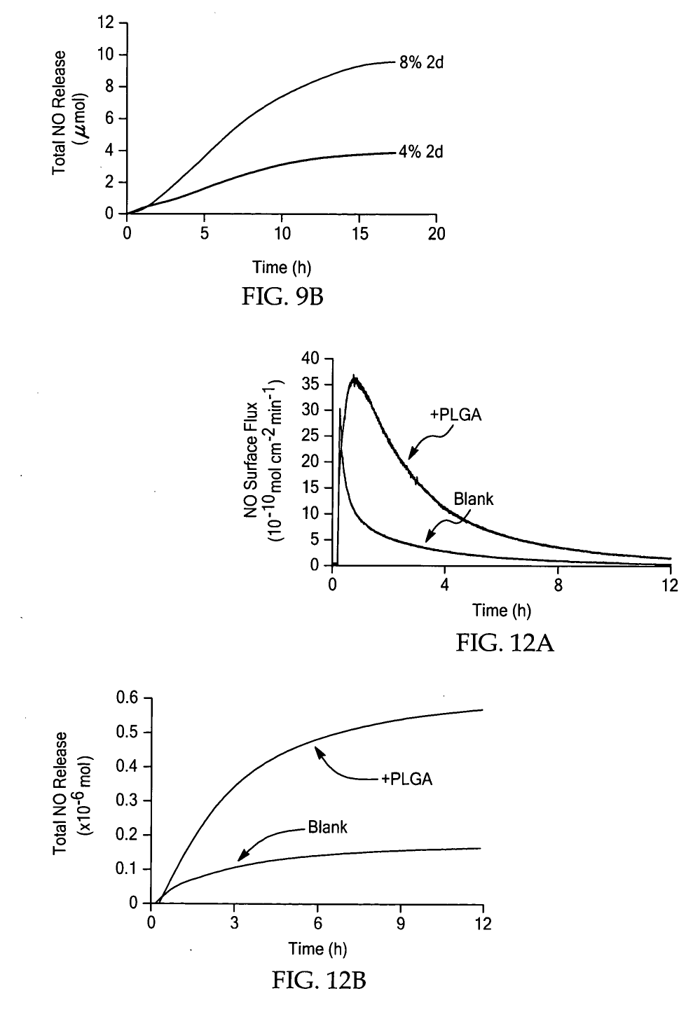

Effect of NO donor amount on NO release. In addition to matrix modifications to alter NO surface flux of materials, changing the amount of lipophilic NO-donor that is incorporated within the polymer matrix may also be employed to alter the rate and total amount of NO released. As shown in FIGS. 9A and PB, doubling the NO donor (compound 2d) in a PVC film, nearly doubles the surface flux and the NO release. This readily allows the preparation of materials with a wide variation of NO surface fluxes from the same polymer matrix.

Stability. One concern with using polymeric materials that contain diazeniumdiolates for medical applications is the stability of the diazeniumdiolates with respect to air and temperature. For example, it has been shown that diazeniumdiolated diaminoalkyltrimethoxylsilane crosslinked polydimethylsiloxane polymer, (DACA/N₂O₂) continuously releases NO at room temperature before being exposed to water, necessitating cold storage (freezer) for optimal stability. For an NO donor material to be used practically, the shelf-life and storage conditions should be suitable to preserve the diazeniumdiolate moiety, thus it may be desirable that these conditions be close to ambient.

As stated previously, the diazeniumdiolate species under investigation remain thermally stable up to about 104° C. under a nitrogen environment. To determine if polymer films containing such diazeniumdiolates would also remain stable, NO release measurements were conducted for films containing 29 wt. % PVC, 60 wt. % plasticizer (DOS), 4.4 wt. % compound 2d and 6.6 wt. % KTpClPB under two storage conditions: a) room temperature under nitrogen and b) ambient conditions. Initial NO release measurements were performed for freshly prepared films. Additional NO release measurements were conducted for both storage conditions after 1, 2 and 4 weeks. Films stored under ambient conditions showed the greatest loss of NO after a 4-week period, achieving only 62% of the theoretical total NO release. The loss of NO may be due in part to the slow decomposition of the diazeniumdiolate within the polymer matrix owing to permeation of water vapor into the polymer film. Although this loss represents a significant percentage of the total NO releasing capability of the film, such films still yield NO surface fluxes higher than that of stimulated endothelial cells (i.e., 4·10⁻¹⁰ mol·cm⁻²·min⁻¹). However, films stored under a dry nitrogen environment at room temperature maintained 99% of their NO release after a 4-week period.

The high thermal stability of 2(a-e) in a nitrogen environment and the stability of these NO-donors embedded into polymer films under appropriate storage conditions, polymer materials prepared with these small molecule diazeniumdiolates and stored properly have the potential to remain shelf stable for extended periods of time. Preliminary Application to Vascular Grafts. Thrombus formation is an important factor potentially limiting the long-term patency of synthetic vascular grafts used for arterial reconstruction and hemodialysis access. It is hypothesized that NO-releasing biopolymers may prolong graft patency by reducing platelet adhesion and hence thrombus formation on the surface of synthetic vascular grafts.

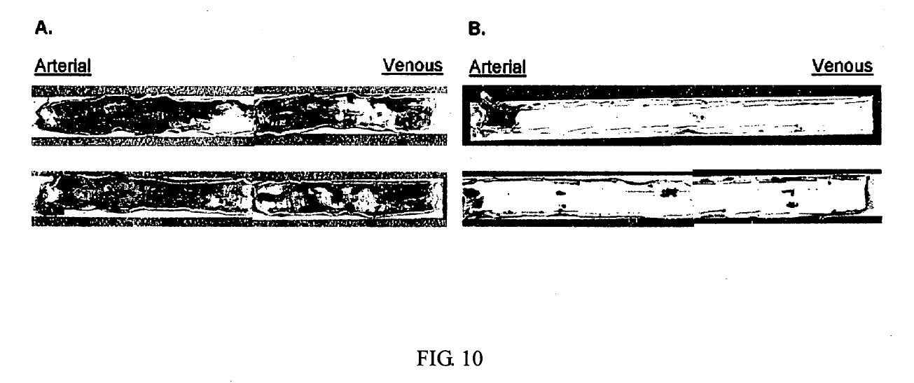

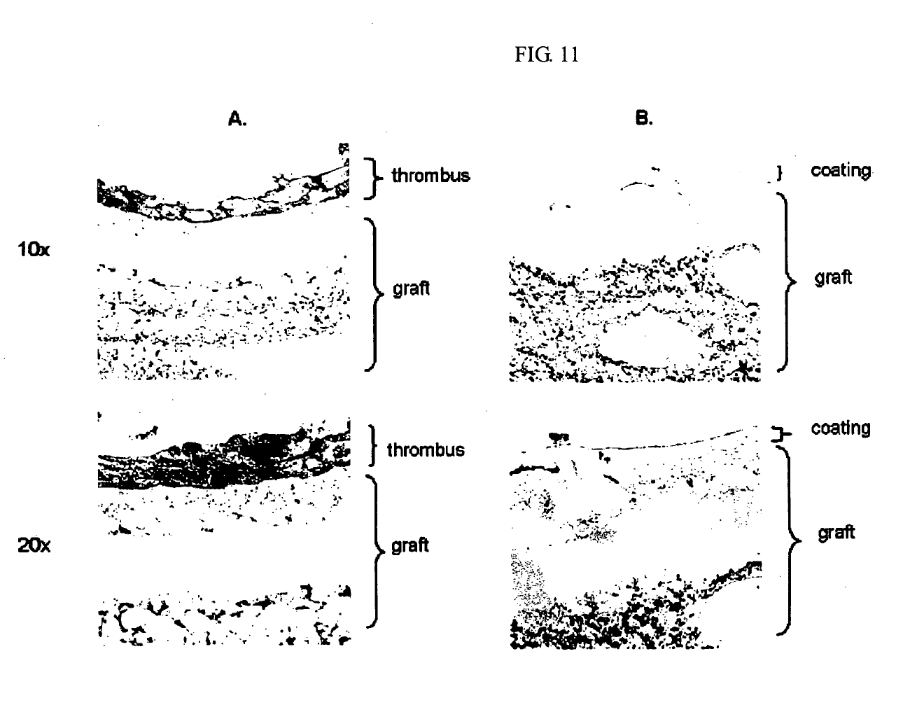

Synthetic vascular access grafts (VECTRATM), a proprietary blend of segmented polyetherurethane and siloxane (20 cm in length and 5 mm in diameter), were coated with compound 2d dispersed within a PVC/DOS matrix with appropriate additives. A sheep model was used for in vivo testing. Arteriovenous grafts connecting the common carotid artery to the ipsilateral external jugular vein were surgically implanted in subcutaneous tunnel in adult sheep. Over a 3-week period, duplex ultrasound and clinical examination were performed to assess graft patency. Grafts were removed at 21 days and underwent gross and histological evaluation. Each control graft (n=2) occluded prior to 21 days and was found to have a mean luminal thrombus-free surface area of 42%. In contrast, the NO-coated grafts (n=2) were patent at 21 days and had a mean luminal thrombus-free surface area of 95%. FIG. 10A clearly shows the gross thrombus formation on the control grafts and FIG. 10B shows the greatly reduced degree of thrombus formation on the NO release grafts. In addition, histological studies, using appropriate stains to highlight different regions, confirm the thrombus adherent at the luminal surface and red blood cell infiltration into upper layers of the control grafts (FIG. 11A). In contrast, the NO release grafts showed minimal thrombus formation and red blood cell infiltration (FIG. 11B).

These results strongly suggest that NO-release biopolymers, prepared with more lipophilic diazeniumdiolates as described herein, may prove effective in reducing thrombus formation on prosthetic vascular grafts as well as other bioprosthetic medical devices.

Experimental Details

Instrumentation. ¹H-NMR spectra were collected on a Varian Mercury.300 or Inova.400 and were referenced to the residual proton solvent resonance. UV-vis spectra were monitored on a Beckman DU 640B spectrophotometer. FT-IR were collected on Perken Elmer Spectrum VX. Thermogravimetric analysis (TGA) was performed on a Perken-Elmer DSC/TGA 7 under nitrogen. Elemental analyses were performed.

Reagents. High molecular weight poly(vinyl chloride) (PVC), dioctyl sebacate (DOS), o-nitrophenyloctyl ether (NPOE), 9-dimethylamino-5-[4-(16-butyl-2,14-dioxo-3,15-dioxaeicosyl)pneynylimino]benzo[a]penoxazine (chromoionophore II) and potassium tetrakis(4-chlorophenylborate) (KTpClPB) were purchased from Fluka (Ronkonkoma, N.Y.). Phosphate buffered saline (PBS), pH 7.4, containing 138 mM NaCl and 2.7 mM KCl was obtained from Sigma (St. Louis, Mo.). N-dodecylamine, N-dodecylamine, N-hexylamine, N-pentylamine, N,N′-dibutyl-1,6-hexanediamine (1d), N,N′-dimethyl-1,6-hexanediamine (1a), adipoyl chloride, triethylamine (Net₃), and lithium aluminum hydride (LiAlH₄) were purchased from Aldrich (Milwaukee, Wis.). N,N′-diethyl-1,6-hexanediamine (1b) and N,N′-dipropyl-1,6-hexanediamine (1c) were purchased from Pfaltz and Bauer (Waterbury, Conn.). Tetrahydrofuran (THF), ethyl acetate, hexane, dichloromethane (CH₂Cl₂), chloroform (CHCl₃), and acetonitrile (CH₃CN) were products of Fisher (Fair Lawn, N.J.). NO was purchased from Matheson Gases. Didodecylhexamethylene diamine (1g), dihexylhexamethylene diamine (1f) and dipentylhexamethylene diamine (1e) were synthesized as described below. All other reagents were analytical reagent grade or better and were used without further modification.

General NO Addition Procedure. The NO-addition process was carried out as described by Hrabie, et al. In brief, a dry parr bottle, equipped with a magnetic stir bar, was charged with the diamine compound dissolved in an appropriate solvent (either CH₃CN or diethyl ether). The reaction vessel was attached to the NO reactor (a modified hydrogenation system), and the headspace purged with argon, up to 1 atm 6 times, to remove air from the connector lines and then up to 80 psi argon 25 times over a 1 hour period. The solution was then charged with NO up to 80 psi. The solution was allowed to stir between 15 and 24 hours, during which time a white precipitate formed. The NO was then released and the headspace purged thoroughly with argon. The NO-adducts were obtained by filtration and washed three times with either CH₃CN or diethyl ether. The products were finally collected and dried under vacuum.

Molar Extinction Coefficients. UV-vis spectra were collected on a Beckman DU 640B spectrophotometer. The samples were prepared by dissolving the NO-adduct in 25 mL of 0.0375M NaOH to make a 0.125 mM diazeniumdiolate solution. The sample was placed into a quartz cuvette and the spectrum was obtained by scanning between 200 nm and 500 nm. The maximum wavelength and absorbance were collected. The molar extinction coefficient was then calculated using Beer's Law: A=Ebc, where A is the absorbance, ε is the molar extinction coefficient, b is the pathlength (1 cm), and c is the concentration.

Kinetics Studies. The half-lives and “apparent” half-lives for the diazeniumdiolates under investigation were determined using chemiluminescence. Between 25 and 100 μL of the respective diazeniumdiolate solution in 10 mM NaOH was injected into the reaction cell containing 3 mL of 100 mM phosphate buffer (pH 7.4) containing 137 mM NaCl and 2.7 mM KCl heated to 37° C. The NO-released from the sample was collected at 0.25 second intervals until baseline NO levels were observed. From the total NO release, the time at which half of the diazeniumdiolate groups had decomposed was determined to be the half-life. The apparent half-lives of the non-water soluble diazeniumdiolate compounds were determined by dissolving the diazeniumdiolate in THF or a mixture of NaOH and MeOH, and then injecting between 25 and 100 μL of the diazeniumdiolate solution into 3 mL deoxygenated PBS buffer. The NO released was measured using chemiluminescence. The “apparent” half-lives were calculated as the time taken to release half of the total diazeniumdiolate moieties.

Preparation of Polymer Films Containing Diazeniumdiolates. Polymer membranes containing the dialkylhexanediamine diazeniumdiolates were prepared by dissolving poly(vinyl chloride) (PVC) and dioctyl sebacate (DOS) totaling 200 mg in 1.5 mL of freshly distilled tetrahydrofuran. The diazeniumdiolates were dispersed within the polymer cocktail via sonication for 10 minutes to obtain a slightly cloudy dispersion of the diazeniumdiolate within the polymer solution. The polymer cocktail was then cast into a 2.5 cm diameter Teflon ring with a Teflon base. The membranes were covered and allowed to cure overnight. Polymer films containing additives were prepared in a similar manner. Smaller disks were cut from the parent films the next morning and measured for their NO release via chemiluminescence.

Polymerization to Form Boc-protected Diamine Copolymer. The following is the synthesis of one of the polymer based NO donors.

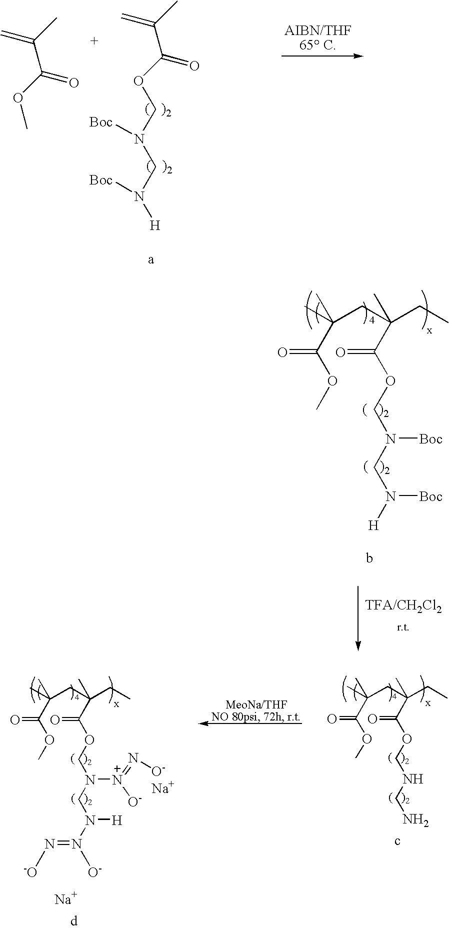

Methacrylic monomer (a), and methyl methacrylate were mixed in a mole ratio (1:4) aiming to achieve copolymer (b) (where the composition of the monomer (a) is about 20 mol %). About 0.22 mmol of the monomer mixture was dissolved in 1 mL of dry THF and placed in a 5 mL reaction vial equipped with a small stirring bar and Teflon seal. An initiator (0.5 mol % of AIBN (2-2′-azo-bis-isobutyrylnitrile)) was added to the mixture. Before heating, the vial was flushed with argon for about 10 minutes and the reactor was sealed and then placed in an oil bath at about 65-70° C. The mixture was stirred for 48 hours at this temperature. The solution was then concentrated to about 0.3 mL, and the polymer was precipitated with 5-10 mL hexane. This dissolving-and-precipitating procedure was repeated three times in order to substantially remove any impurities. The polymer was dried under vacuum overnight. ¹H-NMR was performed afterwards to confirm the structure and actual composition of the copolymer.

General Procedure for Deprotection to Form Diamine Copolymer (c). 35 mg of the copolymer (b) was dissolved in 2 mL of dichloromethane and then 200 μL of TFA (trifluoroacetic acid) was added dropwise. The solution was stirred at room temperature for about 3 hours. The reaction mixture was diluted with 20 mL of dichloromethane, and the organic phase was then washed with sodium bicarbonate, water, and brine, and was dried with sodium sulfate. The solvent was evaporated and the resulting copolymer was dried under vacuum overnight. ¹H-NMR was performed to confirm the structure and actual composition of the copolymer.

General Procedure for NO Loading to Form Polymer-Based NO Donor (d). 20 mg of the deprotected copolymer (c) was dissolved in 3 mL of dry THF and placed in a high pressure reactor with a stirring bar, and flushed with argon for about 10 minutes. 100% excess of sodium methoxide (with respect to free amine sites) was added and the reactor was closed. The reactor was purged with argon again for several times (e.g. ten times) over a 1 hour period and was charged with NO at 80 psi. The reaction mixture was stirred for 72 hours at room temperature. After the reaction was complete, the copolymer was precipitated with dry hexane under argon. The remaining solvent was removed using a vacuum, and the diazeniumdiolated copolymers (d) were examined by UV-Vis spectrum as well as NOA. This resulting copolymer was stored in sealed vials charged with argon in a freezer.

Preparation of Polymer Films Containing Polymer-based NO Donor. Polymer membranes containing the polymer-based NO donor was prepared by dissolving 60.8 mg poly(vinyl chloride) (PVC), 30.4 mg dioctyl sebacate (DOS), and 9.1 mg poly(lactide-co-glycolide) (50:50) (PLGA 50:50) in 1.5 mL of freshly distilled tetrahydrofuran. The polymer-base NO donor was dispersed within the polymer cocktail via sonication for 10 minutes to obtain a slightly cloudy dispersion of the diazeniumdiolate within the polymer solution. A trilayer film configuration is employed to fabricate films. A straight PVC solution (20 mg/mL in THF) was first cast into a 2.5 cm diameter Teflon ring with a Teflon base. Four hours later the polymer cocktail was cast on top of the PVC layer. After another 4 hours, the PVC solution was cast again as a finish layer. The membrane was covered and allowed to cure overnight. Polymer films containing no additives were prepared in a similar manner. Small circular disks with a diameter of 7 mm and a thickness of 150-200 mm were cut from the parent films the next morning and measured for their NO release via chemiluminescence.

NO Release Measurements by Chemiluminescence. All NO measurements were performed using a Sievers Nitric Oxide Analyzer (NOA), model 280. The instrument was calibrated before each experiment using an internal two-point calibration (zero gas and 45 PPM). The flow rate was set to 200 mL/min with a cell pressure of 5.4 torr and an oxygen pressure of 6.0 psig. The measurement was performed by inserting the NO-adducts or polymeric films into a clean, dry, NOA measurement cell, sealing the cell with a rubber septum and collecting a baseline level of nitric oxide. Nitrogen purged PBS buffer was then injected via syringe through a septum into the NOA measurement cell. The NO generated from the sample was removed from the solution via a constant nitrogen purge at 5 mL/min. The data was recorded as a concentration of NO in PPB or PPM. The total amount of NO released (in moles) was determined by multiplying a constant (specific to each instrument, units of mol ppm⁻¹s⁻¹) by the time interval that data was collected and the concentration.

pH Experiments. Two polymer solutions containing 1-hydroxy-2-oxo-3-(-butyl-6-aminohexyl)-3-butyl-1-triazene (compound 2d) were prepared by dissolving 66 mg PVC and 134 mg DOS in 1.5 mL of THF. To one sample, 15.7 mg KTpClPB was added (cocktail 1). 1-hydroxy-2-oxo-3-(N-butyl-6-aminohexyl)-3-butyl-1-triazene (10 mg) was added and dispersed within each of the polymer solutions via sonication. To a 25 μL aliquot (5.8×10⁻⁷ moles diazeniumdiolate) of the cocktail without KTpClPB, 75 μL of a 1:2 PVC/DOS solution and 1.25 μL (4.0×10⁻⁹ moles) of 2 mM KTpClPB was added along with 6.81 μL (1.8×10⁻⁸ moles) of a 1.25 mM Chromoionophore II solution. The 2 mM KTpCIPB solution was prepared by dissolving 4.95 mg of KTpClPB in 5 mL THF, and the 1 mM Chromoionophore II solution was prepared by dissolving 1.47 g Chromoionophore II in 2 mL THF. The aliquot was vortexed and cast onto quartz slides. To a 100 μL (2.3×10⁶ moles) aliquot of the cocktail with KTpClPB, 14.5 μL (1.8×10⁻⁸ moles) Chromoionophore II was added. The solution was vortexed thoroughly and cast onto a quartz slide. The slide was immobilized within a cuvette, 2 mL of PBS added and the spectrum recorded from 400-800 nm. Additional spectra were recorded with time.

Thermal Gravametric Analysis. The TGAs were obtained by increasing the temperature slowly in a nitrogen environment. The data was recorded on Perkin-Elmer DSC/TGA 7.

Synthesis of Discrete Diazeniumdiolates. (Z)-1-{N-Ethyl-N-[6-(N-ethylammoniohexyl)amino]}diazen-1-ium-1,2-diolate (2b) was synthesized by treating N,N′-diethyl-1,6-hexanediamine (Pfaltz and Bauer, 2 mL (9.54 mmol)) in 250 mL CH₃CN with NO for 22 hours: yield 0.152 g (6.8%); mp 118° C.; ¹H NMR (0.01 M NaOD) δ 0.67 (6 H, tt); 1.11 (6 H, s); 1.51 (2 H, s); 2.29 (4 H, t), 2.65 (4 H, t); ¹³C NMR δ 11.1, 13.9, 25.9, 26.0, 26.2, 28.2, 42.8, 48.3, 49.0, 54.1. Anal. Calc'd for C₁₀H₂₄N₄O₂; C, 51.70; H, 10; N, 24.12. Found: C, 51.97; H, 10.43; N, 23.65.

(Z)-1-{N-Propyl-N-[6-(N-propylammoniohexyl)amino]}-diazen-1-ium-1,2-diolate (2c) was synthesized by treating N,N′-dipropyl-1,6,hexanediamine (Pfaltz and Bauer, 2 mL (8.20 mmol)) in 200 mL CH₃CN with NO for 17 hours: yield 0.336 g (15.7%); mp 120° C.; ¹H NMR (0.01 M NaOD) δ 0.68 (6 H, t); 0.1.12 (6 H, s); 1.56 (4 H s); 2.29 (4 H, m), 2.71 (4 H, m); ¹³C NMR δ 11.1, 11.3, 19.8, 22.0, 25.8, 26.0, 26.2, 28.4, 48.7, 50.8, 54.2, 56.0 Anal. Calc'd for C₁₂H₂₈N₄O₂: C, 55.35; H, 10.84; N, 21.52. Found: C, 55.99; H, 10.94; N, 20.93.

(Z)-1-{N-Butyl-N-[6-(N-butylammoniohexyl)amino]}-diazen-1-ium-1,2-diolate (2d) was synthesized by treating N,N′-dibutyl-1,6-hexanediamine (Aldrich, 1.5 mL (5.40 mmol)) in 250 mL CH₃CN with NO for 16.5 hours; yield 0.504g (32.7%); mp 120° C.; ¹H NMR (0.01 M NaOD) δ 0.72 (6 H, m), 1.15 (12 H, m), 1.26 (6, m), 2.34 (4 H, m), 2.72 (4 H, t); ¹³C NMR δ 15.78, 15.86, 22.20, 22.50, 28.37, 28.65, 28.93, 30.65, 30.94, 33.39, 50.87, 51.09, 56.46, 56.63. Anal. Calc'd for C₁₄H₃₂N₄O₂: C, 58.30; H, 11.18; N, 19.42. Found: C, 58.38; H, 11.18; N, 18.66.

(Z)-1-{N-Pentyl-N-[6-(N-pentylammoniohexyl)amino]}-diazen-1-ium-1,2-diolate (2e) was synthesized by treating N,N′-dipentyl-1,6-hexanediamine (1e), 0.7321 (2.86 mmol)) in 20 mL CH₃CN with NO for 22 hours: yield 0.420 g (46.4%); mp 113° C.; ¹H NMR (CD₃OD) δ 0.92 (6 H, t); 1.33 (16 H, m); 1.52 (4 H, m); 2.53 (4 H, m), 2.84 (4 H, m); Anal. Calc'd for C₁₈H₃₆N₄O₂: C, 60.72; H, 11.47; N, 17.70. Found C, 61.10; H, 11.49, N, 17.22

Hexanedioic acid bis-pentylamide (3e) was synthesized by equipping a dry 250 mL 3-neck flask with a condenser, addition funnel, stir bar and inlet/outlet and charging it with N-pentylamine (15 mL, 0.129 mol) and triethylamine (30 mL, 0.215 mol) in CHCl₃ (125 mL). Adipoyl chloride (8.8 mL, 0.0605 mol) in CHCl₃ was added dropwise over 20 minutes, during which time a white precipitate formed. After 3 hours, the solvent was removed under vacuum to give a white solid. The solid was stirred in hot water for 30 min and filtered. The solid was washed with additional water followed by acetonitrile. The white solid was collected and dried under vacuum. Yield: 12.33 g (71%); mp 152° C.; ¹H NMR δ 0.89 (6 H, t), 1.3 (8 H, m), 1.45-1.50 (4 H, m), 1.66 (4 H, m), 2.1-2.3 (4 H, m), 3.2 (4 H, m), 5.7 (2 H, s); ³C NMR δ 14.1, 22.0, 25.3, 29.2, 37.3, 40.0, 172. Anal. Calc'd for C₁₆H₃₂N₂O₂: C, 67.56; H, 11.34; N, 9.85. Found: C, 67.88; H, 11.65; N, 9.92. MS (Cl)=[M+H]⁺=285.2532.

N,N′-Dipentylhexane-1,6-diamine (1e) was synthesized by charging a dry 250 mL 3-neck flask equipped with a condenser, stir bar, and N₂ inlet/outlet with lithium aluminum hydride (LiAlH₄) (2.82 g, 74.3 mmol) in dry THF (150 mL). Hexanedioic acid bis-pentylamide (3e) (4.32 g, 15.1 mmol) was carefully added as solid portions over 30 min. The reaction was heated to reflux for 16 hours. The reaction flask was placed in an ice bath, and the LiAlH was carefully quenched with 100 mL of 1 M sodium potassium tartrate. The mixture was filtered, and the solid residue was washed with ethyl acetate (100 mL). The aqueous phase was extracted with ethyl acetate several times. The organic portions were combined and dried over MgSO₄. The solvent was removed to give an oil, which was purified via vacuum distillation 0.3 mmHg at 120° C. The colorless liquid solidified upon standing. The white solid was dried under vacuum. Yield: 2.26 g (50%); ¹H NMR δ 0.88 (6 H, t), 1.27-1.43 (12 H, m), 1.49 (8 H, m), 2.58 (8 H, tt). ¹³C NMR δ 14.1 (2). 22.6 (2), 27.4 (2), 39.9 (2), 30.5 (2), 30.9 (2), 50.5 (4). MS (Cl with ammonia)=[M+H]⁺=257.3 Anal. Calc'd for C₁₆H₃₆H₂: C, 74.93; H, 14.15; N, 10.92. Found: C, 70.45; H, 10.14; N, 13.12.

Discussion. The potential advantages of the novel, more lipophilic diazeniumdiolates described herein for preparing NO release polymeric coatings are numerous. First, the amount of NO-donor incorporated into thin polymeric films may be substantially easily controlled, thereby giving various release profiles (e.g., NO fluxes), for a given application. With these new materials, thin coatings with high NO loading may be prepared for circumstances where polymer thickness is limited (e.g., coatings for catheters), and NO may be stored until needed and then delivered under physiological conditions. Finally, potential by-products resulting from diazeniumdiolate decomposition are more confined to the polymer matrix, due in part to the increased lipophilicity of these species, thereby generally reducing the toxicity threat to biological systems.

Many medical devices suffer from blood compatibility issues including platelet adhesion and activation on their surfaces. The ability to synthesize and incorporate NO donors into hydrophobic polymers to prevent such a response is desirable. Currently, systemic anticoagulant treatments are required to minimize the risk of thrombus formation, but this approach may have the increased risk of uncontrolled bleeding elsewhere in the body associated with it. The use of new lipophilic diazeniumdiolates discussed hereinabove may be useful in developing polymeric coatings with greatly improved thromboresistivity, thereby minimizing the need for systemic anticoagulation. The results discussed herein using NO releasing arterial grafts in a sheep model strongly support the potential biomedical utility of this approach.

To further illustrate embodiment(s) of the present invention, the following examples are given. It is to be understood that these examples are provided for illustrative purposes and are not to be construed as limiting the scope of embodiment(s) of the present invention.

### EXAMPLES 1-6

The following are structural illustration examples of the dispersion of discrete diazeniumdiolates [1]; covalent attachment of discrete diazeniumdiolates to a linear polymer backbone [2]; covalent attachment of discrete diazeniumdiolates to a pendent polymer chain [3]; and the dispersion of protected discrete diazeniumdiolates [4].

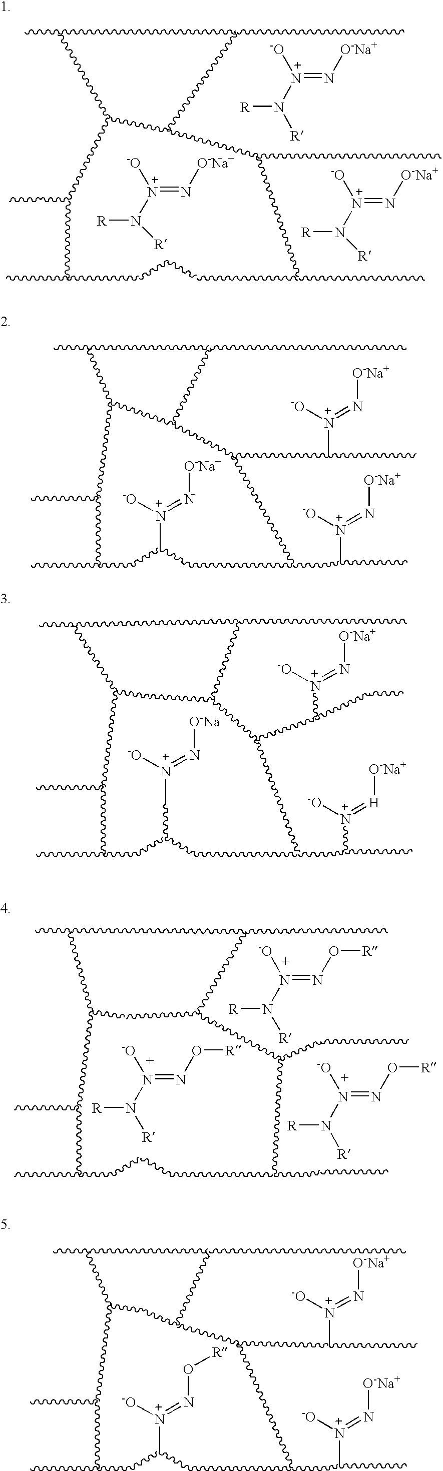

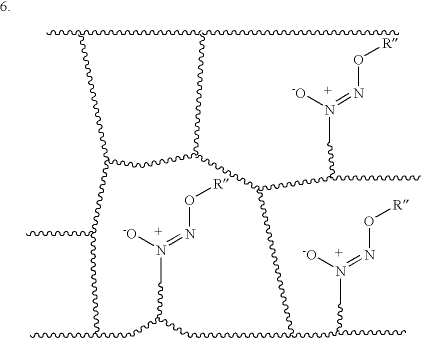

Examples 5 and 6 are non-limitative embodiments of covalently attached protected diazeniumdiolates to a linear polymer backbone [5] and covalently attached protected diazeniumdiolates to a pendant polymer backbone [6].

In these examples, sodium ions are used as a representative example of a stabilizing countercation. However, it is to be understood that other cations or intramolecular stabilization (a hydrogen bond species) may be used to stabilize the diazeniumdiolates.

While several embodiments have been described in detail, it will be apparent to those skilled in the art that the disclosed embodiments may be modified. Therefore, the foregoing description is to be considered exemplary rather than limiting.

TABLE 1
Characteristics of Parent N-N′-dialkylhexamethlyenediamine
Structures

| Compound | R |
|---|---|
| 1a | —CH₃ |
| 1b | —CH₂CH₃ |
| 1c | —(CH₂)₂CH₃ |
| 1d | —(CH₂)₃CH₃ |
| 1e | —(CH₂)₄CH₃ |
| 1f | —(CH₂)₅CH₃ |
| 1g | —(CH₂)₁₁CH₃ |

TABLE 2
Characteristics of N-Diazeniumdiolate Structures (all measurements were n > 3)

| | | | | | Ratio | ε | T_{NO loss} |
|---|---|---|---|---|---|---|---|
| Compound | R | LogP^{a} | k(s⁻¹) | t_{1/2}^{b} (s) | Diamine:NO | (M⁻¹ cm⁻¹) | (° C.) |
| 2a | —CH₃ | 0.97 | 0.010 ± 0.0001 | 67.2 ± 0.7 | 1:2.0 ± 0.1 | 7250 | 104 |
| 2b | —CH₂CH₃ | 2.03 | 0.0020 ± 0.0001 | 347 ± 26 | 1:2.10 ± 0.04 | 8640 | 104 |
| 2c | —(CH₂)₂CH₃ | 3.09 | 0.0022 ± 0.0001 | 319 ± 23 | 1:2.00 ± 0.05 | 7868 | 104 |
| 2d | —(CH₂)₃CH₃ | 4.15 | 0.0025 ± 0.0002 | 297 ± 34 | 1:2.0 ± 0.1 | 7818 | 104 |
| 2e | —(CH₂)₄CH₃ | 5.21 | 0.0025 ± 0.0001 | 279 ± 31 | 1:1.9 ± 0.1 | 8045 | 104 |
| 2f | —(CH₂)₅CH₃ | 6.26 | ^{c} | | | | 104 |
| 2g | —(CH₂)₁₁CH₃ | 12.6 | ^{c} | 660^{d} | | | 104 |

^{a}Octanol/water partition coefficient calculated using ChemDraw;
^{b}“Apparent” t_{1/2} and t_{1/2} for diazeniomdiolates under investigation in PBS buffer at 37° C. and pH 7.4;
^{c}Unable to determine based on the lack of air stability; and
^{d}Measured using an NO-selective electrochemical sensor in a nitrogen environment.

1. A biocompatible material, comprising:
a polymer matrix having at least one of a discrete nitric oxide adduct and a polymeric nitric oxide adduct covalently bound thereto; and
at least one of an anionic site additive, an acidic site additive, and a site adapted to produce an acidic site additive within the polymer matrix.

2. The biocompatible material as defined in claim 1 wherein the polymer matrix comprises a hydrophobic polymer.

3. The biocompatible material as defined in claim 1 wherein the polymer matrix comprises at least one of poly(vinyl chloride), silicone rubbers, polyurethanes, polymethacrylates, polyacrylates, polycaprolactone, copolymers thereof, polylactide, polyglycolide, poly(lactide-co-glycolide), or mixtures thereof

4. The biocompatible material as defined in claim 1 wherein the anionic site additive is selected from potassium tetrakis-4-(chloro)phenyl borate, sodium cholate, carboxylated poly(vinyl chloride), dinonylnaphthalene sulfonate, phosphatidylglycerol, L-pbosphatidic acid, L-glycerol 3-phosphoric acid, phosphoglycerides, phosphatidylinsitol, sodium salts, potassium salts, cholesterols, steroid derivatives, lipids, phosphatidyl chlorine, prostaglandins, lipophilic fatty acids, lipophilic sugars, and mixtures thereof.

5. The biocompatible material as defined in claim 1, further comprising a plasticizer.

6. The biocompatible material as defined in claim 5 wherein the plasticizer is selected from dioctyl sebacate, isopropyl palmitate, isopropyl isosterate, diisooctyl phitalate, o-nitrophenyloctyl ether, and mixtures thereof.

7. The biocompatible material as defined in claim 1 wherein the polymer matrix further comprises chromoionophores.

8. The biocompatible material as defined in claim 1 wherein the discrete nitric oxide adduct is selected from discrete N-diazeniumdiolates, nitrosothiols, organic nitrates, metal-nitrosyls, C-based diazeniumdiolates, and mixtures thereof.

9. The biocompatible material as defined in claim 8 wherein the discrete N-diazeniumdiolates is selected from anionic diazeniumdiolates stabilized by metal cations, zwitterionic diazeniumdiolates, and mixtures thereof.

10. The biocompatible material as defined in claim 1 wherein the site adapted to produce an acidic site additive is selected from a biodegradable polymer and a biodegradable copolymer.

11. The biocompatible material as defined in claim 10 wherein the at least one of the biodegradable polymer and the biodegradable copolymer is selected from poly(lactide-co-glycolide)polylactide, polyglycolide, polycaprolactone, poly(lactide-co-caprolactone), and mixtures thereof.

12. The biocompatible material as defined in claim 1 wherein the polymer matrix contains the anionic site additive.

13. The biocompatible material as defined in claim 1 wherein the polymer matrix is selected from carboxylated poly(vinyl chloride), a sodium salt of carboxylated poly(vinyl chloride)(PVC—COOM, polymethacrylic acid, poly(anetholesulfonic acid, sodium salt), and mixtures thereof.

14. The biocompatible material as defined in claim 1 wherein the polymer matrix comprises:
a base polymer layer;
a top polymer layer disposed on the base polymer layer; and
at least one layer intermediate the bane polymer layer and the top polymer layer, the at least one intermediate layer including the at least one of the discrete nitric oxide adduct, the polymeric nitric oxide adduct, the anionic site additive, the acidic site additive, the site adapted to produce an acidic site additive, a polymeric material, and mixtures thereof.

15. The biocompatible material as defined in claim 1 wherein the polymeric nitric oxide adduct is selected from of diazeniumdiolated silicone rubbers, diazeniumdiolated methacrylates, diazeniumdiolated polyurethanes, diazeniumdiolated poly(vinyl chloride), and mixtures thereof.

16. The biocompatible material as defined in claim 1 wherein the acidic site additive is selected from a polymer having an acidic group, a copolymer having an acidic group, and mixtures thereof.

17. A biocompatible material, comprising:
a nitric oxide adduct; and
at least one of an anionic site additive, an acidic site additive, and a site adapted to produce an acidic site additive within the biocompatible material.

18. The biocompatible material as defined in claim 17, further comprising a hydrophobic polymer.

19. The biocompatible material as defined in claim 17, further comprising at least one of poly(vinyl chloride), silicone rubbers, polyurethanes, polymethacrylates, polyacrylates, polycaprolactone, polylactide, polyglycolide, poly(lactide-co-glycolide), copolymers thereof, and mixtures thereof.

20. The biocompatible material as defined in claim 17 wherein the anionic site additives selected from potassium tetrakis-4-(chloro)phenyl borate, sodium cholate, carboxylated poly(vinyl chloride), dinonylnaphthalene sulfonate, phosphatidylglycerol, L-phosphatidic acid, L-glycerol 3-phosphoric acid, phosphoglycerides, phosphoatidylinsitol, sodium salts, potassium salts, cholesterols, steroid derivatives, lipids, phosphatidyl chlorine, prostaglandins, lipophilic fatty acids, lipophilic sugars, and mixtures thereof.

21. The biocompatible material as defined in claim 17, further comprising a plasticizer.

22. The biocompatible material as defined in claim 21 wherein the plasticizer is selected from of dioctyl sebacate, isopropyl palmitate, isopropyl isosterate, diisooctyl phthalate, o-nitrophenyloctyl ether, and mixtures thereof.

23. The biocompatible material as defined in claim 17 wherein the nitric oxide adduct is selected from, discrete N-diazeniumdiolates, protected N-diazeniumdiolates, nitrosothiols, oric nitrates, metal-nitrosyls, C-based diazeniumdiolates, and mixtures thereof.

24. The biocompatible material as defined in claim 17 wherein the site adapted to produce an acidic site additive is selected from biodegradable polymer and a biodegradable copolymer.

25. The biocompatible material as defined in claim 24 wherein the at least one of the biodegradable polymer and the biodegradable copolymer is selected from poly(lactide-co-glycolide)polylactide, polyglycolide, polycaprolactone, poly(lactide-co-caprolactone), and mixtures thereof.

26. The biocompatible material as defined in claim 17 wherein the biocompatible material contains the anionic site additive.

27. The biocompatible material as defined in claim 26, further comprising at least one of carboxylated poly(vinyl) chloride, a sodium salt of carboxylated poly(vinyl)chloride (PVC—COOH), polymethacrylic acid, poly(anetholesulfonic acid, sodium salt), and mixtures thereof.

28. The biocompatible material as defined in claim 17 wherein the acidic site additive is selected from a polymer having an uncapped acidic end group, a copolymer having an uncapped acidic end group, a polymer having an acidic group attached to at least one of its backbone and pendant side chains, a copolymer having an acidic group attached to at least one of its backbone and pendant side chains, and mixtures thereof.

29. A biocompatible material, comprising:
a base polymer layer;
a top polymer layer;
a first layer intermediate the base polymer layer and the top polymer layer, the first intermediate layer including a nitric oxide adduct; and
at least one second layer intermediate to the base polymer layer and the top polymer layer, the at least one second layer including at least one of the nitric oxide adduct, am anionic site additive, an acidic site additive, and a site adapted to produce an acidic site additive.

30. A method for making an NO-releasing polymer, comprising the steps of:
providing a biocompatible polymer having at least one of an of anionic site additive, an acidic site additive, and a site adapted to produce an acidic site additive therein; and
dispersing an amount of an NO adduct into the biocompatible polymer to form an NO-releasing polymer;
wherein the at least one of the anionic site additive, the acidic site additive and the site adapted to produce an acidic site additive is adapted to at least one of increase, prolong, and control the release of NO.

31. The method as defined in claim 30 wherein the anionic site additive is in the biocompatible polymer.

32. The method as defined in claim 30 wherein the biocompatible polymer is selected from carboxylated poly(vinyl chloride), a sodium salt of carboxylated poly(vinyl chloride), polymethacrylic acid, poly(anetholesulfonic acid, sodium salt), and mixtures thereof.

33. The method as defined in claim 30, further comprising the step of adding the anionic site additive to the biocompatible polymer prior to providing the biocompatible polymer.

34. The method as defined in claim 33 wherein the anionic site additive is selected from potassium tetrakis-4-(chloro)phenyl borate, sodium cholate, carboxylated poly(vinyl chloride), dinonylnaphthalene sulfonate, phosphatidylglycerol, L-phosphatidic acid, L-glycerol 3-phosphoric acid, phosphoglycerides, phosphatidylinsitol, sodium salts, potassium salts, cholesterols, steroid derivatives, lipids, phosphatidyl chlorine, prostaglandins, lipophilic fatty acids, lipophilic sugars, and mixtures thereof.

35. The method as defined in claim 30 wherein the site adapted to produce an acidic site additive is selected from poly(lactide-co-glycolide)polylactide, polyglycolide, polycaprolactone, poly(lactide-co-caprolactone), and mixtures thereof.

36. The method as defined in claim 30 wherein the acidic site additive is selected from a polymer having an uncapped acidic end group, a copolymer having an uncapped acidic end group, a polymer having an acidic group attached to at least one of its backbone and pendant side chains, a copolymer having an acidic group attached to at least one of its backbone and pendant side chains, and mixtures thereof.

37. A method for making an NO-releasing polymer, comprising the steps of:
providing a biocompatible polymer having at least one of an anionic site additive, an acidic site additive, and a site adapted to produce an acidic site additive therein; and
covalently attaching an amount of a discrete NO adduct to the biocompatible polymer to form an NO-releasing polymer.

38. The method as defined in claim 37 wherein the discrete NO adduct is selected from anionic diazeniumdiolates stabilized by metal cations and zwitterionic diazeniumdiolates.

39. A biocompatible material, comprising:
a polymer matrix having a discrete diazeniumdiolate dispersed therein; and
means for at least one of increasing, prolonging, and controlling NO release rates from the discrete diazeniumdiolate.

40. A thromboresistant device that releases NO at a blood-contacting surface thereof, the device comprising:
a base layer including a first polymer;
an NO-releasing layer including a second polymer, the NO-releasing layer having at least one of a discrete NO-releasing diazeniumdiolate group covalently attached thereto and at least one of an anionic site additive, an acidic site additive, and a site adapted to produce an acidic site additive therein; and
a coating of a biocompatible polymer, the coating providing the blood-contacting surface.

41. A biocompatible material, comprising:
a polymer matrix having at least one of a nitric oxide adduct and a polymeric nitric oxide adduct covalently bound thereto, the at least one of the nitric oxide adduct and the polymeric nitric oxide adduct having a benign protecting group attached thereto, and the benign protecting group adapted to be removed from the at least one nitric oxide adduct and the polymeric nitric oxide adduct at least one of before and during NO release; and
at least one of an anionic site additive, an acidic site additive, and a site adapted to produce an acidic site additive within the polymer matrix, the at least one of the anionic site additive, the acidic site additive, and the site adapted to produce an acidic site additive adapted to at least one of increase, prolong, and control NO release rates from the at least one of the nitric oxide adduct and the polymeric nitric oxide adduct.

42. The biocompatible material as defined in claim 41 wherein the site adapted to produce an acidic site additive is selected from poly(lactide-co-glycolide)polylactide, polyglycolide, polycaprolactone, poly(lactide-co-caprolactone), and mixtures thereof.

43. The biocompatible material as defined in claim 41 wherein the acidic site additive is selected from a polymer having an acidic group, a copolymer having an acidic group, and mixtures thereof.

44. The biocompatible material as defined in claim 41 wherein the anionic site additive is selected from potassium tetrakis-4-(chloro)phenyl borate, sodium cholate, carboxylated poly(vinyl chloride), dinonylnaphthalene sulfonate, phosphatidylglycerol, L-phosphatidic acid, L-glycerol 3-phosphoric acid, phosphoglycerides, phosphatidylinsitol, sodium salts, potassium salts, cholesterols, steroid derivatives, lipids, phosphatidyl chlorine, prostaglandins, lipophilic fatty acids, lipophilic sugar, and mixtures thereof.

45. A biocompatible material, comprising:
a polymer matrix having at least one of a nitric oxide adduct and a polymeric nitric oxide adduct covalently bound thereto, the at least one of the nitric oxide adduct and the polymeric nitric oxide adduct capable of releasing NO, the at least one of the nitric oxide adduct and the polymeric nitric oxide adduct having a non-benign protecting group attached thereto, and the non-benign protecting group adapted to be removed from the at least one nitric oxide adduct and the polymeric nitric oxide adduct before NO release; and
at least one of an anionic site additive, an acidic site additive, and a site adapted to produce an acidic site additive within the polymer matrix, the at least one of the anionic site additive, the acidic site additive, and the site adapted to produce an acidic site additive adapted to at least one of increase, prolong, and control NO release rates from the at least one of the nitric oxide adduct and the polymeric nitric oxide adduct.

46. The biocompatible material as defined in claim 45 wherein tie site adapted to produce an acidic site additive is selected from poly(lactide-co-glycolide)polylactide, polyglycolide, polycaprolactone, poly(lactide-co-caprolactone), and mixtures thereof.

47. The biocompatible material as defined in claim 45 wherein the acidic site additive is selected from a polymer having an acidic group, a copolymer having an acidic group, and mixtures thereof.

48. The biocompatible material as defined in claim 45 wherein the anionic site additive is selected from potassium tetrakis-4-(chloro)phenyl borate, sodium cholate, carboxylated poly(vinyl chloride), dinonylnaphthalene sulfonate, phosphatidylglycerol, L-phosphatidic acid, L-glycerol 3-phosphoric acid, phosphoglycerides, phosphatidylinsitol, sodium salts, potassium salts, cholesterols, steroid derivatives, lipids, phosphatidyl chlorine, prostaglandins, lipophilic fatty acids, lipophilic sugars, and mixtures thereof.

49. The biocompatible material as defined in claim 3 wherein the polymer matrix comprises a polyvinyl chloride.

50. The biocompatible material as defined in claim 49 wherein the acidic site additive comprises a polymer having an uncapped acidic end group.

51. The biocompatible material as defined in claim 49, further comprising at least one of polylactide, polyglycolide, or poly(lactide-co-glycolide).

52. The biocompatible material as defined in claim 1 wherein the at least one of the anionic site additive, the acidic site additive, and the site adapted to produce an acidic site additive is adapted to at least one of increase, prolong, and control NO release rates from the at least one of the discrete nitric oxide adduct and the polymeric nitric oxide adduct.

53. The thromboresistant device as defined in claim 40 wherein the NO-releasing layer is disposed on the base layer.

54. The thromboresistant device as defined in claim 53 wherein the coating is disposed on the NO-releasing layer.
